(19) United States
(12) Patent Application Publication
(10) Pub. No.: US 2007/0073115 A1
(43) Pub. Date: Mar. 29, 2007

(54) Apparatus and method for measuring concentration of blood component using terahertz

(75) Inventors: In Duk Hwang, Suwon-si (KR); Kyung Ho Kim, Yongin-si (KR)

Correspondence Address: STAAS & HALSEY LLP, SUITE 700, 1201 NEW YORK AVENUE, N.W., WASHINGTON, DC, 20005, US

(73) Assignee: Samsung Electronics Co., Ltd., Suwon-si (KR)

(21) Appl. No.: 11/373,218

(22) Filed: Mar. 13, 2006

(30) Foreign Application Priority Data
Sep. 23, 2005 (KR) 10-2005-0088515

(51) Int. Cl. A61B 5/00

(52) U.S. Cl. 600/309

(57) ABSTRACT

An apparatus for measuring blood component concentration by using a terahertz wave, the apparatus comprising: a terahertz light source unit generating a terahertz wave in a predetermined range of THz, and emitting the generated terahertz wave to a specific region of a living body to measure blood component concentration; a terahertz generation controller controlling the generation of the terahertz wave in the terahertz light source unit; a terahertz detector detecting the intensity of light reflected from or penetrating into the specific region; a concentration analyzer analyzing the blood component concentration by using the detected light intensity; and a concentration indicator indicating the analyzed blood component concentration.

### CROSS-REFERENCE TO RELATED APPLICATION

This application claims the benefit of Korean Patent Application No. 10-2005-88515, filed on Sep. 23, 2005, in the Korean Intellectual Property Office, the disclosure of which is incorporated herein by reference.

### BACKGROUND OF THE INVENTION

1. Field of the Invention

The present invention relates to an apparatus and method for measuring a blood component concentration by using a terahertz wave, and more particularly, to an apparatus and method for measuring a blood component concentration by emitting a terahertz wave to a specific region of a living body and detecting the intensity of light reflected from or penetrating into the specific region.

2. Description of Related Art

A terahertz wave is positioned in the band between the microwave portion of electronics and the far infrared portion of optics on a radio spectrum as illustrated in FIG. 1. Accordingly, a terahertz wave simultaneously maintains the characteristics of microwaves and optics.

Because of the aforementioned characteristics, a terahertz wave may be very useful in obtaining a high density material penetrating image or spectroscopy. However, the technology of generating and measuring an electromagnetic wave in the frequency band shown has been slow in development because of the difficulty of embodying capable equipment.

However, currently, the combination of a laser having the pulse width of millions of femtoseconds and a photoconductive material having a carrier lifetime of less than picoseconds has made it possible to generate and measure a terahertz wave. Accordingly, various technologies utilizing the characteristics of terahertz waves are being developed.

A terahertz wave is capable of penetrating into material into which a microwave and optics cannot penetrate. Accordingly, various apparatuses are being developed by using the above characteristics.

The developed apparatuses, by using the characteristics of a terahertz wave, are usually used for spectroscopy with respect to various materials and semiconductor diagnostic systems, and for analyzing material in a nondestructive and non-contact manner.

Also, a vibration energy level of most materials having large molecules is in the terahertz wave band. Accordingly, a terahertz band may be used to reveal the characteristics of various gases and plasma.

At the same time, a terahertz wave is very sensitive to water. The degree of absorption with respect to water is very high, about 230 cm⁻¹ with respect to 1 terahertz. Accordingly, a terahertz wave is almost incapable of penetrating into a sample such as a tissue containing much water. A terahertz wave may be used for medical imaging systems by using the characteristics described above.

Also, while a terahertz wave is capable of penetrating into almost all materials like an X-ray, the terahertz wave is safer than an X-ray. Also, a terahertz wave is capable of revealing a chemical composition of a hidden material via an imaging system. In this instance, the imaging system uses the properties that a compound reacts to radioactivity in a unique way. Imaging technology using a terahertz wave is being further developed because of the aforementioned characteristics of a terahertz wave.

U.S. Patent Publication No. U.S. 2003/0149346 discloses an image generating apparatus and method in which an image is generated by emitting one pulse in the frequency band between 25 GHz and 100 THz to the surface of a sample, detecting a signal reflected from the sample, and indicating structure information about the sample by using a depth function from the surface of the sample.

The conventional image generating apparatus and method needs to synchronize a pulse time by using a reference synchronization signal to generate an image. Also, the conventional image generating apparatus and method may generate an image by detecting a signal reflected from a sample and using structure information about the sample by using a depth function from the surface of the sample. However, it has no relation to analysis of the component concentration of the sample.

Korean Patent Publication No. 2002-0002214 discloses an apparatus for inspecting the impurity concentration of a semiconductor and a method of inspecting the same in which an analytic method of calculating oxygen concentration, nitrogen concentration and carbon concentration of semiconductor materials is implemented. The conventional apparatus for inspecting the impurity concentration of a semiconductor and a method for inspecting the same has to be provided with a measurement device in a terahertz time-domain obtaining a spectrum transmittance from a time series waveform of the field intensity of a transmitted pulse.

However, only when a terahertz wave is transmitted to a semiconductor, the conventional method and apparatus for inspecting impurities of a semiconductor material can inspect the concentration of impurities such as oxygen concentration, nitrogen concentration, and carbon concentration of a semiconductor.

A method for measuring the blood component concentration of a patient involves taking blood from a blood vessel of the patient's finger and analyzing the components contained within the blood. However, the conventional method for measuring blood component concentration by direct blood collecting may make a patient endure pain since the blood has to be directly taken to measure the blood component concentration. Also, when a blood collecting device is not sterilized, the above conventional method may infect a patient with other diseases or provide an inaccurate measurement value.

As described above, a terahertz wave has various characteristics and technologies using the same have been widely dissemination. However, until now, a method and apparatus for measuring blood component concentration by using a terahertz wave has not been developed.

Accordingly, an apparatus and method for noninvasively measuring blood component concentration by using a terahertz wave are needed.

### BRIEF SUMMARY

An aspect of the present invention provides an apparatus and method for noninvasively measuring blood component concentration by using a terahertz wave.

An aspect of the present invention also provides an apparatus and method for detecting the intensity of light reflected by a terahertz wave emitted to a specific region of a living body and measuring blood component concentration.

According to an aspect of the present invention, there is provided an apparatus for measuring blood component concentration by using a terahertz wave, the apparatus including: a terahertz light source unit generating a terahertz wave in a predetermined range of THz, and emitting the generated terahertz wave to a specific region of a living body to measure blood component concentration; a terahertz generation controller controlling the generation of the terahertz wave in the terahertz light source unit; a terahertz detector detecting the intensity of light reflected from or penetrating into the specific region; a concentration analyzer analyzing the blood component concentration by using the detected light intensity; and a concentration indicator indicating the analyzed blood component concentration.

According to another aspect of the present invention, there is provided a method for measuring blood component concentration by using a terahertz wave, the method including the steps of: generating a terahertz wave in a predetermined range of THz; emitting the generated terahertz wave to a specific region of a living body to measure blood component concentration; detecting the intensity of light reflected from or penetrating into the specific region; analyzing the blood component concentration by using the detected light intensity; and indicating the analyzed blood component concentration.

According to another aspect of the present invention, there is provided a method of non-invasively measuring a concentration of a blood component, including: directing an electromagnetic wave in a terahertz band to a specific region of a subject; detecting a change in a characteristic of the electromagnetic wave reflected from or penetrating into the specific region; analyzing the blood component concentration using the detected change; and indicating the analyzed blood component concentration.

According to another aspect of the present invention, there is provided a non-invasive blood component concentration measuring apparatus, including: an electromagnetic wave directing unit directing an electromagnetic wave in a terahertz band to a specific region of a subject; a detector detecting a change in a characteristic of the electromagnetic wave reflected from or penetrating into the specific region; an analyzer analyzing the blood component concentration using the detected change; and a concentration indicator indicating the analyzed blood component concentration.

Additional and/or other aspects and advantages of the present invention will be set forth in part in the description which follows and, in part, will be obvious from the description, or may be learned by practice of the invention.

### BRIEF DESCRIPTION OF THE DRAWINGS

The above and/or other aspects and advantages of the present invention will become apparent and more readily appreciated from the following detailed description, taken in conjunction with the accompanying drawings of which:

FIG. 1 is a diagram illustrating a general radio spectrum;

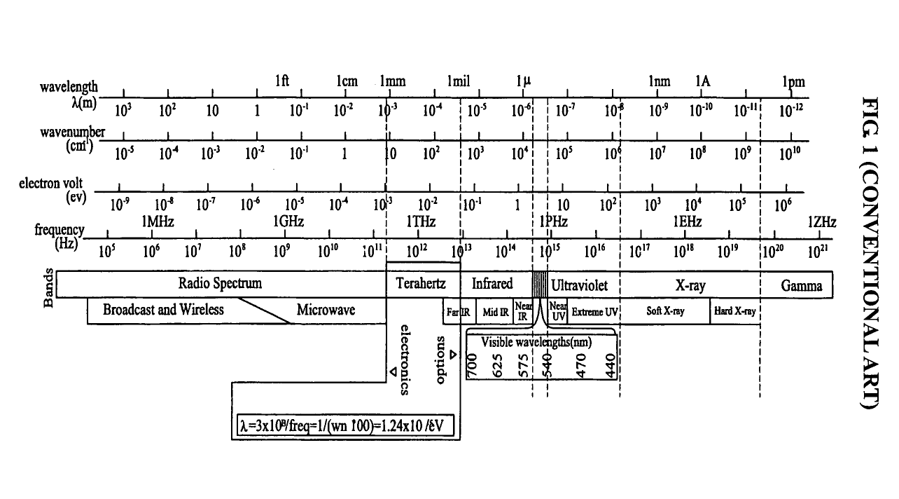

FIG. 2 is a diagram illustrating a configuration of an apparatus for measuring a blood component concentration by using a terahertz wave according to an embodiment of the present invention;

FIG. 3 is a diagram illustrating an example of an absorption spectrum with respect to blood components in the blood component concentration measuring apparatus of FIG. 2; and

FIG. 4 is a flowchart illustrating the process of measuring a blood component concentration by using a terahertz wave according to an embodiment of the present invention.

### DETAILED DESCRIPTION OF EMBODIMENTS

Reference will now be made in detail to embodiments of the present invention, examples of which are illustrated in the accompanying drawings, wherein like reference numerals refer to the like elements throughout. The embodiments are described below in order to explain the present invention by referring to the figures.

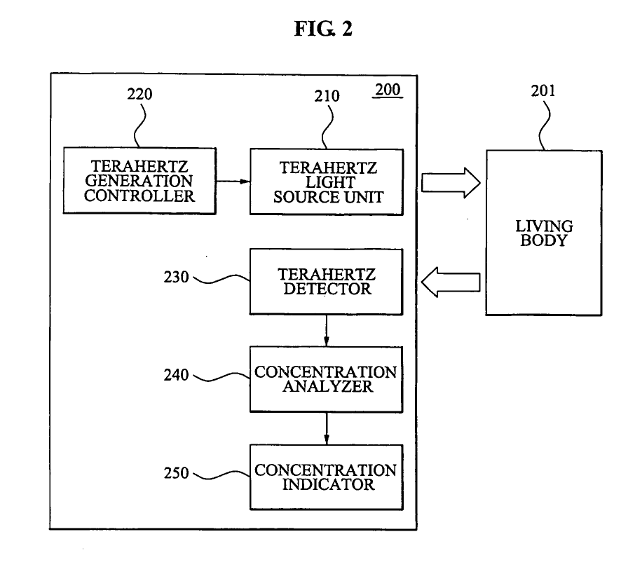

FIG. 2 is a diagram illustrating a configuration of an apparatus for measuring a blood component concentration by using a terahertz wave according to an embodiment of the present invention.

Referring to FIG. 2, a blood component concentration measuring apparatus 200 includes a terahertz light source unit 210, a terahertz generation controller 220, a terahertz detector 230, a concentration analyzer 240, and a concentration indicator 250.

The terahertz light source unit 210 generates a terahertz wave in a predetermined range of THz. As a light source generating a terahertz wave in a predetermined range of THz via a semiconductor laser is miniaturized, the terahertz light source unit 210 may be provided in a portable device by making the blood component concentration measuring apparatus 200 in a small size. Accordingly, a user may more easily measure the blood component concentration as necessary while carrying the blood component concentration measuring apparatus 200 installed in a small sized portable device. The terahertz light source unit 210 may generate a terahertz wave in a range between 0.3 and 10 THz.

The terahertz light source unit 210 emits the generated terahertz wave to a specific region of a living body 201 to measure blood component concentration. For example, the terahertz light source unit 210 may emit the generated terahertz wave to the specific region of the living body 201, such as a subject's finger, arm, and the like.

The terahertz generation controller 220 controls the generation of the terahertz wave in the terahertz light source unit 210. The terahertz generation controller 220 may control the terahertz light source unit 210 to generate the terahertz wave as a pulse or continuous signal.

The terahertz detector 230 detects the intensity of light reflected from or penetrating into the specific region of the living body 201. The intensity of light indicates the characteristics of the change of diffusion, absorption, and polarization of the terahertz wave according to the change of the blood component concentration. The terahertz detector 230 may detect the change of permittivity (rate of propagation) when the terahertz wave penetrates into the specific region of the living body 201.

The blood component concentration measuring apparatus 200 according to the present embodiment directly measures the intensity of light reflected from or penetrating into the specific region of the living body 201. Accordingly, time synchronization with the light source does not need to be performed separately.

The blood component concentration measuring apparatus 200 according to the present embodiment may be more useful as a reflection type than as a penetration type in the case of performing measurement with respect to the living body 201.

The concentration analyzer 240 analyzes blood component concentration by using the detected light intensity. Namely, the concentration analyzer 240 may analyze the blood component concentration by using the characteristics of the change of diffusion, absorption, and polarization of the detected light intensity, that is, the terahertz wave according to the change of the blood component concentration.

The concentration analyzer 240 may analyze blood component concentration such as glucose, cholesterol, albumin, hemoglobin, bilirubin, and the like, by using the detected light intensity.

Also, the concentration analyzer 240 may analyze the blood component concentration by using the change of detected permittivity. Namely, the concentration analyzer 240 may detect the change of permittivity of the detected terahertz wave, that is, the light intensity according to the change of blood component concentration, and analyze the blood component concentration.

The concentration indicator 250 indicates the analyzed blood component concentration. The concentration indicator 250 may classify and indicate the analyzed blood component concentration according to the blood components. The concentration indicator 250 may indicate the analyzed blood component concentration in a form of a graph. Also, the concentration indicator 250 may indicate said each analyzed blood component concentration in a numerical value.

Accordingly, the blood component concentration measuring apparatus according to the present embodiment enables inspection of various blood component concentrations, such as glucose, cholesterol, albumin, hemoglobin, bilirubin, and the like. In this instance, the blood component concentration is displayed via the concentration indicator 250.

As described above, the blood component concentration measuring apparatus 200 according to an embodiment of the present invention emits a terahertz wave to the specific region of the living body 201, detects the intensity of light reflected from or penetrating into the specific region of the living body 201, analyzes blood component concentration, and indicates the analyzed blood component concentration. Accordingly, the blood component concentration measuring apparatus 200 may noninvasively measure blood component concentration and may not need to take blood via a blood collecting device.

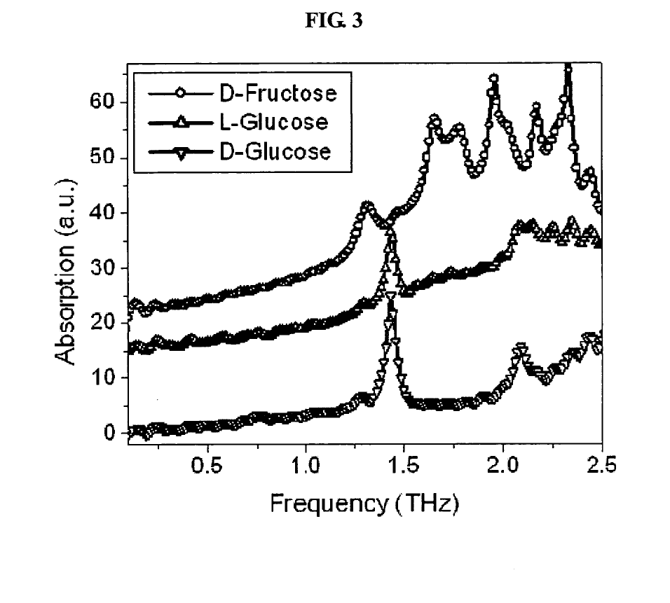

FIG. 3 is a diagram illustrating an example of an absorption spectrum with respect to blood components in the blood component concentration measuring apparatus of FIG. 2.

Referring to FIG. 3, an absorption spectrum with respect to D-fructose, L-glucose, and D-glucose as an example of blood components exists in a terahertz band.

A blood component concentration measuring apparatus according to the present invention generates a terahertz wave in a range between 0.3 and 10 THz, emits the generated terahertz wave to a specific region of a living body, and detects the change of absorbance of fructose or glucose in the terahertz band. The blood component concentration measuring apparatus may measure the concentration in the blood of components such as fructose or glucose by using the detected absorbance.

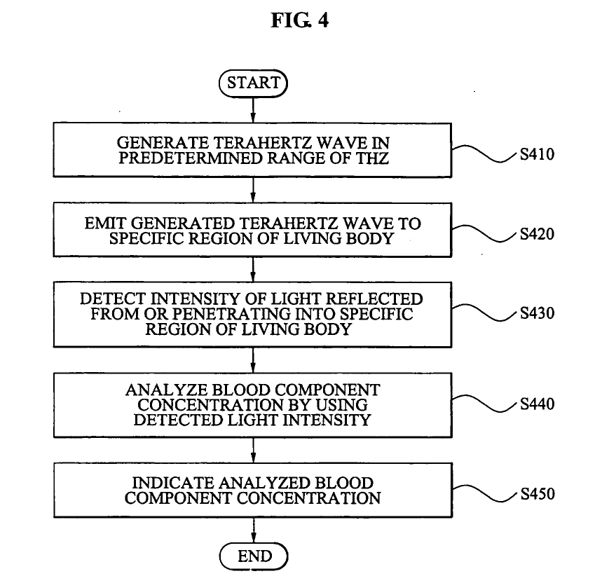

FIG. 4 is a flowchart illustrating the process of measuring blood component concentration by using a terahertz wave according to an embodiment of the present invention. This method is hereafter described with concurrent reference to FIGS. 2 and 4 for ease of explanation only. It is to be understood that apparatuses of configurations other than that shown in FIG. 2 may execute this method.

Referring to FIGS. 2 and 4, in operation S410, the blood component concentration measuring apparatus 200 generates a terahertz wave in a predetermined range of THz via the terahertz light source unit 210. In this operation S410, the blood component concentration measuring apparatus 200 may generate a terahertz wave in a range between 0.3 and 10 THz via the terahertz light source unit 210. Also, in this operation S410, the blood component concentration measuring apparatus 200 may control the terahertz light source unit 210 to generate the terahertz wave in a pulse or continuous signal via the terahertz generation controller 220.

In operation S420, the blood component concentration measuring apparatus 200 emits the generated terahertz to a specific region of a living body to measure blood component concentration via the terahertz light source unit 210. As an example, the specific region of the living body may include a finger or an arm.

In operation S430, the blood component concentration measuring apparatus 200 detects the intensity of light reflected from or penetrating into the specific region of the living body via the terahertz detector 230. In this operation S430, the blood component concentration measuring apparatus 200 may detect the change of permittivity via the terahertz detector 230 when the terahertz wave penetrates into the specific region of the living body.

In operation S440, the blood component concentration measuring apparatus 200 analyzes blood component concentration by using the detected light intensity. The blood component is a component contained in blood such as glucose, cholesterol, albumin, hemoglobin, bilirubin, or the like. In this operation S440, the blood component concentration measuring apparatus 200 may analyze the blood component concentration by using the change of detected permittivity via the concentration analyzer 240.

In operation S450, the blood component concentration measuring apparatus 200 indicates the analyzed blood component concentration via the concentration indicator 250.

According to the above-described embodiments of the present invention, since the blood component concentration of a living body is non-invasively measured by using a terahertz wave, the danger of infection or pain endured by a patient caused by blood-collecting may be reduced.

Also, according to the above-described embodiments of the present invention, terahertz light source is miniaturized by using a semiconductor laser and a blood component concentration measuring apparatus using a terahertz wave is provided. Accordingly, ease of use and utility may be increased.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. An apparatus for measuring a blood component concentration using a terahertz wave, the apparatus comprising:
a terahertz light source unit generating a terahertz wave in a predetermined range of THz, and directing the generated terahertz wave to a specific region of a living body to measure blood component concentration;
a terahertz generation controller controlling the generation of the terahertz wave by the terahertz light source unit;
a terahertz detector detecting an intensity of light reflected from or penetrating into the specific region;
a concentration analyzer analyzing the blood component concentration using the detected light intensity; and
a concentration indicator indicating the analyzed blood component concentration.

2. The apparatus of claim 1, wherein the terahertz wave is in a range between 0.3 and 10 THz.

3. The apparatus of claim 1, wherein the concentration analyzer analyzes the concentration of at least one of glucose, cholesterol, albumin, hemoglobin and bilirubin.

4. The apparatus of claim 1, wherein the terahertz generation controller controls the terahertz light source unit to generate the terahertz wave as a pulse or a continuous signal.

5. The apparatus of claim 1, wherein:
the terahertz detector further detects a change of permittivity of the terahertz wave when the terahertz wave penetrates into the specific region, and
the concentration analyzer analyzes the blood component concentration using the detected change of permittivity.

6. The apparatus of claim 1, wherein the intensity of light indicates a change in diffusion, absorption, or polarization characteristics of the terahertz wave.

7. A method of measuring a blood component concentration using a terahertz wave, the method comprising:
generating a terahertz wave in a predetermined range of THz;
directing the generated terahertz wave to a specific region of a living body to measure the blood component concentration;
detecting an intensity of light reflected from or penetrating into the specific region;
analyzing the blood component concentration using the detected light intensity; and
indicating the analyzed blood component concentration.

8. The method of claim 7, wherein the terahertz wave is in a range between 0.3 and 10 THz.

9. The method of claim 7, further comprising:
detecting a change of permittivity of the terahertz wave when the terahertz wave penetrates into the specific region, and
analyzing the blood component concentration using the detected change of permittivity.

10. The method of claim 7, wherein the terahertz wave is a pulse or a continuous signal.

11. The apparatus of claim 7, wherein the intensity of light indicates a change in diffusion, absorption, or polarization characteristics of the terahertz wave.

12. A method of non-invasively measuring a concentration of a blood component, comprising:
directing an electromagnetic wave in a terahertz band to a specific region of a subject;
detecting a change in a characteristic of the electromagnetic wave reflected from or penetrating into the specific region; and
analyzing the blood component concentration using the detected change; and indicating the analyzed blood component concentration.

13. The method of claim 12, further comprising: detecting a change in at least one propagation characteristic of the electromagnetic wave, when the electromagnetic wave penetrates into the specific region; and
analyzing the blood component concentration using the detected change in the at least one propagation characteristic.

14. A non-invasive blood component concentration measuring apparatus, comprising:
an electromagnetic wave directing unit directing an electromagnetic wave in a terahertz band to a specific region of a subject;
a detector detecting a change in a characteristic of the electromagnetic wave reflected from or penetrating into the specific region;
an analyzer analyzing the blood component concentration using the detected change; and
a concentration indicator indicating the analyzed blood component concentration.

15. The method of claim 14, wherein the detector detects a change in at least one propagation characteristic of the electromagnetic wave, when the electromagnetic wave penetrates into the specific region; and
the analyzer analyzes the blood component concentration using the detected change in the at least one propagation characteristic.
